(19) United States
(12) Patent Application Publication
(10) Pub. No.: US 2022/0183681 A1
(43) Pub. Date: Jun. 16, 2022

(54) PURSE STRING SUTURE PASSER DEVICE

(71) Applicant: Covidien LP, Mansfield, MA (US)

(72) Inventors: Justin Thomas, New Haven, CT (US); Saumya Banerjee, Collinsville, CT (US); Matthew A. Dinino, Newington, CT (US); Garrett P. Ebersole, Hamden, CT (US); Jacob C. Baril, Norwalk, CT (US); Roy J. Pilletere, Middletown, CT (US)

(21) Appl. No.: 17/522,089

(22) Filed: Nov. 9, 2021

Related U.S. Application Data
(60) Provisional application No. 63/125,620, filed on Dec. 15, 2020.

(51) Int. Cl. A61B 17/06; A61B 17/11

(52) U.S. Cl. CPC A61B 17/06066; A61B 17/11; A61B 2017/06009; A61B 2017/1142; A61B 2017/0609; A61B 17/06004

(57) ABSTRACT

A purse string suture passing device includes a clamp member and a suture passer housing assembly. The clamp member is dimensioned to be received within a guide channel of the suture passer housing assembly and includes two jaws that each define a longitudinal passage. The suture passer housing assembly includes two needles that are supported within the guide channel and are received within the longitudinal passages of the clamp member as the clamp member is inserted through the guide channel. Each of the needles receives a suture passer that includes a distal portion that is configured to engage a suture after the clamp member is received within the guide channel. When the suture is attached to the suture passers, the clamp member can be removed from the guide channel to apply a purse string suture to the tissue.

### CROSS-REFERENCE TO RELATED APPLICATION

This application claims the benefit of and priority to U.S. Provisional Patent Application No. 63/125,620, filed Dec. 15, 2020, the entire contents of which is incorporated by reference herein.

### FIELD

This technology is generally related a device for passing a suture through tissue, and more particularly, to a device for passing a suture through tissue to form a purse string in the tissue.

### BACKGROUND

Surgical anastomosis is an artificial connection of two vessel portions made by a surgeon and is required when a diseased portion of a vessel, e.g., artery, vein, or intestine, is removed from the vessel and remaining vessel portions must be rejoined. Typically, a circular stapling device is used to rejoin the vessel portions. In such a procedure, a purse string suture is formed in an end of each of the vessel portions and the purse string sutures are tightened to secure the ends of the vessel portions about an anvil shaft of an anvil of the circular stapling device and about an anvil retainer of the circular stapling device.

A purse string suture is a suture that is stitched into an end portion of each of the vessel portions and used as a drawstring to tighten the end portions about the anvil shaft and/or anvil retainer of a circular stapling device. Once the end portion of each of the vessel portions is tightly secured about the anvil shaft and/or the anvil retainer of the circular stapling device, the anvil of the circular stapling device is approximated with a staple cartridge of the circular stapling device to clamp the vessel portions to each other between the anvil and a staple cartridge of the circular stapling device. The circular stapling device is then fired to secure the vessel portions together with staples and to remove excess tissue from within a lumen defined by the joined vessel portions.

The process of forming a purse string suture in each end portion of the vessel portions is time consuming and increases the amount of time a patient must remain under anesthesia. This increases the trauma the patient must endure and increases the likelihood of infection. In some procedures, such as lower anterior resection, access to the end portions of the vessel portion is limited making the formation of a purse string suture in the tissue even more difficult and time consuming.

A continuing need exists in the surgical arts for a device for quickly forming a purse string in the end portions of the vessel portions to be joined during an anastomosis procedure to reduce the time required to perform a surgical procedure.

### SUMMARY

This disclosure is directed to a purse string suture passer device that includes a clamp member and a housing assembly that can quickly and easily form a purse string suture in tissue about a vessel. The clamp member is configured to be clamped about tissue and inserted into the housing assembly to position suture passers through the tissue that is clamped in the clamp member. A suture can be attached to the suture passers when the clamp member is received within the housing assembly and the clamp member can be removed from the housing assembly to place the purse string suture in the tissue.

Aspects of this disclosure are directed to a purse string suture passer device that includes a clamp member and a housing assembly. The clamp member includes a first jaw, a second jaw, and a hinge coupling the first jaw to the second jaw. The first jaw and the second jaw are movable in relation to each other about the hinge such that the clamp member is movable between open and clamped positions. Each of the first and second jaws includes a clamping surface having a sinusoidal configuration including hills and valleys. When the clamp member is in the clamped position, the hills of the first jaw are aligned with the valleys of the second jaw and the valleys of the first jaw are aligned with the hills of the second jaw. Each of the hills of the first and second jaws defines a longitudinal through bore that extends though the hills to define a longitudinal passage through each of the first and second jaws. The housing assembly includes a body, first and second hollow needles, and first and second suture passers. The body has a grip portion and first and second guide portions that extend distally from the grip portion. The first and second guide portions are spaced from each other to define a guide channel between the guide portions that is dimensioned to receive the clamp member when the clamp member is in the clamped position. The first and second hollow needles are fixedly secured to and extend distally from the grip portion within the guide channel. The first suture passer is positioned within the first hollow needle and the second suture passer is positioned within the second hollow needle. Each of the first and second suture passers includes a distal hook portion that defines a recess. The first hollow needle is aligned with the longitudinal passage in the first jaw and the second longitudinal needle is aligned with the longitudinal passage in the second jaw such that when the clamp member is received within the guide channel defined between the first and second guide portions, the first hollow needle extends through the first jaw and the second hollow needle extends through the second jaw.

Other aspects of this disclosure are directed to a clamp member including a first jaw, a second jaw, and a hinge coupling the first jaw to the second jaw. The first jaw and the second jaw are movable in relation to each other about the hinge such that the clamp member is movable between open and clamped positions. Each of the first and second jaws includes a clamping surface that has a sinusoidal configuration and includes hills and valleys. The hills of the first jaw are aligned with the valleys of the second jaw and the valleys of the first jaw are aligned with the hills of the second jaw when the clamp member is in the clamped position. Each of the hills of the first and second jaws defines a longitudinal through bore that extends though the hills to define a longitudinal passage through each of the first and second jaws.

Other aspects of the disclosure are directed to a suture passer housing assembly that includes a body, first and second hollow needles, and first and second suture passers. The body has a grip portion and first and second guide portions that extend distally from the grip portion. The first and second guide portions are spaced from each other to define a guide channel between the first and second guide portions that is dimensioned to receive a clamp member. The first and second hollow needles are fixedly secured to and extend distally from the grip portion within the guide channel. The first suture passer is positioned within the first hollow needle and the second suture passer is positioned within the second hollow needle. Each of the first and second suture passers includes a distal hook portion that defines a recess.

In aspects of this disclosure, the first and second suture passers are movable within the first and second needles, respectively, from an intermediate position in which the recesses defined by the distal hook portions are covered by the needle to an advanced position in which the recesses defined by the distal hook portions are uncovered and positioned distally of the needle.

In some aspects of this disclosure, the first and second suture passers are movable within the first and second needles, respectively, from the intermediate position to a retracted position in which the distal portions of the first and second needles are positioned distally of the first and second suture passers.

In certain aspects of this disclosure, the distal portion of each of the first and second suture passers includes a sharpened distal end that is configured to pierce tissue.

In aspects of this disclosure, the housing assembly includes first biasing members positioned within the grip portion to urge the first and second suture passers towards the intermediate position.

In some aspects of this disclosure, the housing assembly includes an actuator that is supported on the grip portion and is movable from a retracted position to an advanced position to move the first and second suture passers from their intermediate positions to their advanced positions.

In certain aspects of this disclosure, each of the first and second guide portions includes an inner guide surface that defines the guide channel.

In aspects of this disclosure, each of the guide portions includes guide walls that are positioned on opposite sides of the guide surface to define the guide channel.

In some aspects of this disclosure, the guide surfaces of the first and second guide portions are angled towards each other in a proximal direction.

In certain aspects of this disclosure, a coupling member is secured to the proximal portion of each of the first and second suture passers.

In aspects of this disclosure, each of the coupling members defines a cavity and the first biasing members are positioned within cavity.

In some aspects of this disclosure, the coupling members are movable in relation to the first and second suture passers between retracted and advanced positions.

In aspects of this disclosure, the hinge is a living hinge that is integrally formed with the first and second jaws.

In some aspects of this disclosure, each of the first and second jaws has a rectangular cross-sectional configuration.

Other features of the disclosure will be appreciated from the following description.

### BRIEF DESCRIPTION OF DRAWINGS

Various aspects of a purse string suture passer device are described herein below with reference to the drawings, wherein:

FIG. 1 is a side perspective view of a purse string suture passer device according to aspects of the disclosure with a tissue clamp of the purse string suture passer device separated from a housing of the purse string suture passer device;

FIG. 2 is an enlarged view of the indicated area of detail shown in FIG. 1;

FIG. 3 is an enlarged view of the indicated area of detail shown in FIG. 1;

FIG. 4 is an exploded perspective view of the housing of the purse string suture passer device shown in FIG. 1;

FIG. 5 is an enlarged view of the indicated area of detail shown in FIG. 1;

FIG. 6 is an enlarged view of a distal end of the housing of the purse string suture passer device shown in FIG. 1 with a suture passer member in an intermediate position;

FIG. 7 is a cross-sectional view taken along section line 7-7 of FIG. 1;

FIG. 8 is an enlarged view of the indicated area of detail shown in FIG. 7;

FIG. 9 is an enlarged view of the indicated area of detail shown in FIG. 7;

FIG. 10 is a cross-sectional view taken along section line 10-10 of FIG. 7;

FIG. 11 is an enlarged view of the indicated area of detail shown in FIG. 10;

FIG. 12 is an enlarged view of the indicated area of detail shown in FIG. 10;

FIG. 13 is a cross-sectional view taken along section line 13-13 of FIG. 1;

FIG. 14 is a cross-sectional view taken through the tissue clamp of the purse string suture passer device shown in FIG. 1 with the tissue clamp clamped about tissue;

FIG. 15 is a cross-sectional view taken through the tissue clamp and the housing of the purse string suture passer device shown in FIG. 1 with the tissue clamp clamped about tissue and spaced from a distal end of the housing;

FIG. 16 is a side perspective view of the tissue clamp and the housing of the purse string suture passer device shown in FIG. 15 with the tissue clamp clamped about tissue and received partly within the distal end of the housing;

FIG. 17 is a cross-sectional view taken along section line 17-17 of FIG. 16;

FIG. 18 is an enlarged view of the indicated area of detail shown in FIG. 17;

FIG. 19 is a perspective view of the distal end of the suture passer member and needle of the housing of the purse string suture passer device shown in FIG. 18 with the suture passer member is retracted within the needle

FIG. 20 is an enlarged view of the indicated area of detail shown in FIG. 17;

FIG. 21 is a cross-sectional view taken through the purse string suture passer device shown in FIG. 17 with the tissue clamp received within the housing and the suture passer in an intermediate position within the needle;

FIG. 22 is an enlarged view of the indicated area of detail shown in FIG. 21;

FIG. 23 is a side perspective view of the tissue clamp and the housing of the purse string suture passer device shown in FIG. 16 with the tissue clamp clamped about tissue and received within the distal end of the housing and the suture passer member in an advanced position;

FIG. 24 is a cross-sectional view taken along section line 24-24 of FIG. 23;

FIG. 25 is an enlarged view of the indicated area of detail shown in FIG. 24;

FIG. 26 is an enlarged view of the indicated area of detail shown in FIG. 23;

FIG. 27 is a side perspective view of the of the purse string suture passer device shown in FIG. 24 with the clamp member removed from the housing and a purse string suture positioned within the tissue;

FIG. 28 is a cross-sectional view taken through the tissue with the purse string suture positioned within the tissue; and

FIG. 29 is a side perspective view of an anvil assembly of a surgical stapling device positioned within the tissue and the purse string suture cinched about an anvil center rod of the anvil assembly.

### DETAILED DESCRIPTION

The disclosed purse string suture passer device will now be described in detail with reference to the drawings in which like reference numerals designate identical or corresponding elements in each of the several views. However, it is to be understood that aspects of the purse string suture passer device disclosed are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure. In addition, directional terms such as front, rear, upper, lower, top, bottom, distal, proximal, and similar terms are used to assist in understanding the description and are not intended to limit the disclosure.

In this description, the term “proximal” is used generally to refer to that portion of the device that is closer to a clinician, while the term “distal” is used generally to refer to that portion of the device that is farther from the clinician. In addition, the term “clinician” is used generally to refer to medical personnel including doctors, nurses, and support personnel.

Aspects of this disclosure are directed to a purse string suture passer device for quickly placing a purse string suture in tissue prior to performing an anastomosis procedure. The purse string suture passing device includes a clamp member and a suture passer housing assembly. The clamp member is configured to clamp tissue into which a purse string suture is to be placed. The clamp member is dimensioned to be received within a guide channel of the suture passer housing assembly and includes two jaws that each define a longitudinal passage. The suture passer housing assembly includes two needles that are supported within the guide channel and are received within the longitudinal passages of the clamp member as the clamp member is inserted through the guide channel. Each of the needles receives a suture passer that includes a distal portion that is configured to engage a suture after the clamp member is received within the guide channel. When the suture is attached to the suture passers, the clamp member can be removed from the guide channel to apply a purse string suture to the tissue.

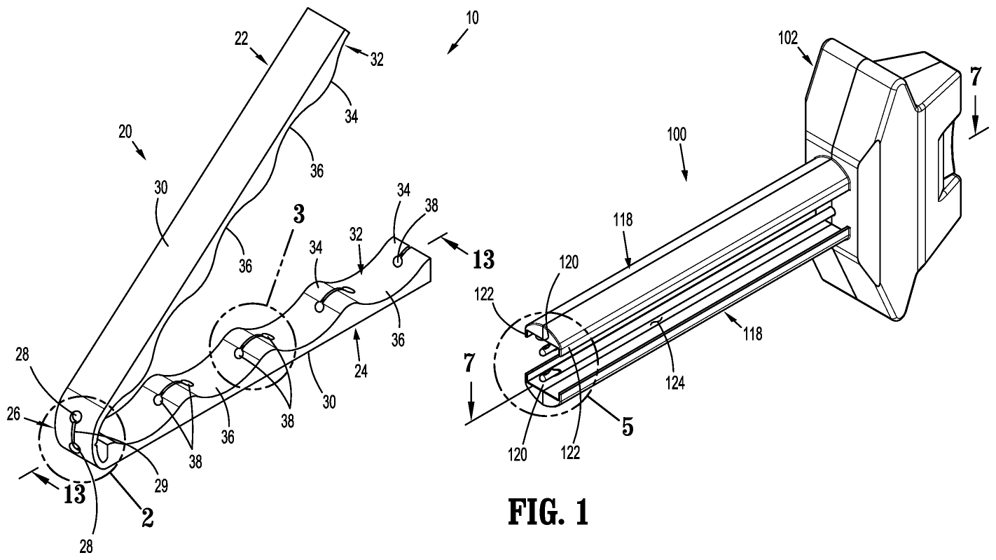

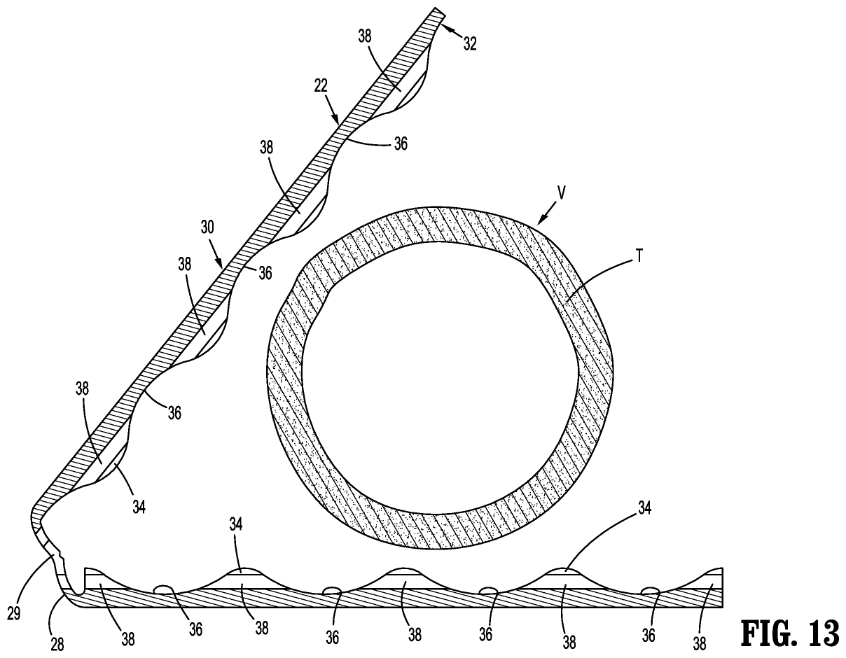

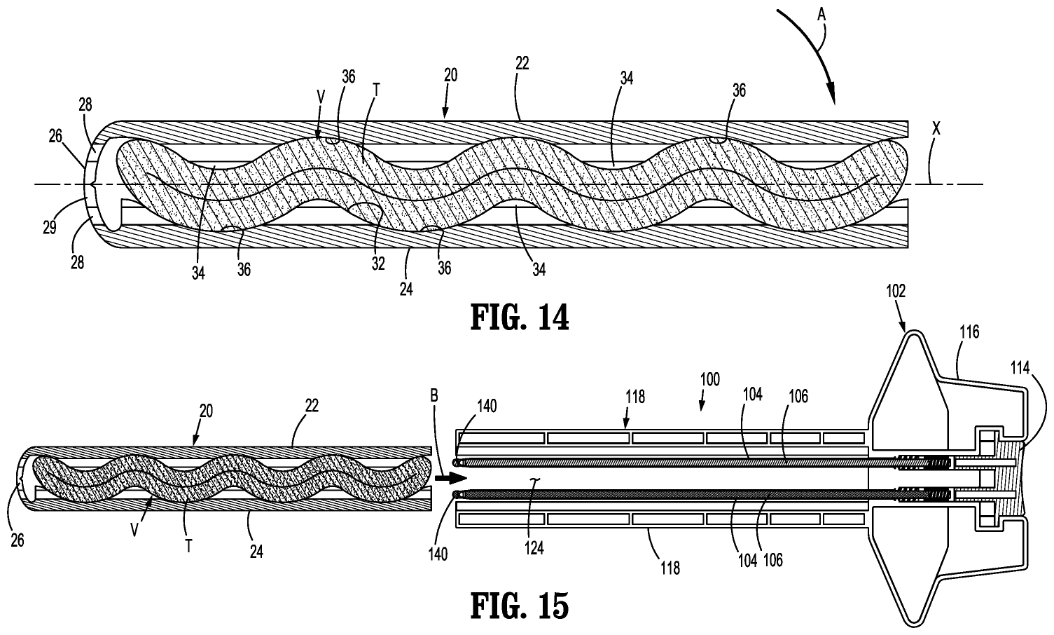

FIG. 1 illustrates aspects of a purse string suture passer device shown generally as passer device 10. The passer device 10 includes a clamp member 20 and a suture passer housing assembly shown generally as housing assembly 100. The clamp member 20 defines a longitudinal axis “X” (FIG. 14) and is movable between an open position (FIG. 13) and a clamped position (FIG. 14) to clamp tissue between jaws of the clamp member 20. In the clamped position, the clamp member 20 is received within the housing assembly 100 to retain the clamp member 20 in the clamped position and to position tissue “T” for receipt of a purse string suture as described in detail below.

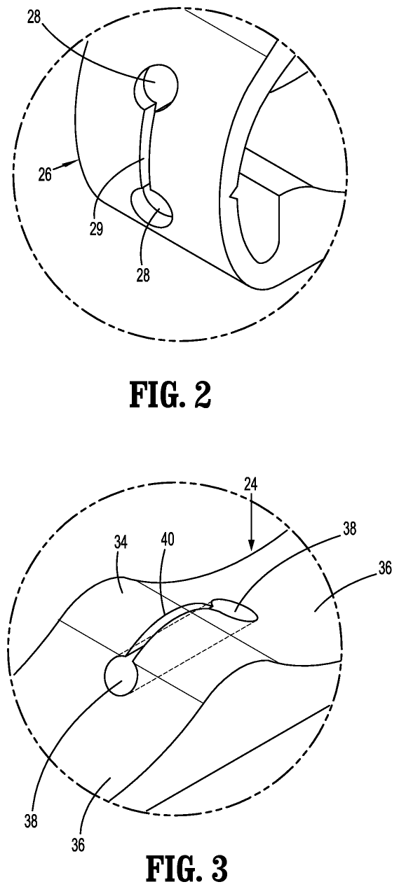

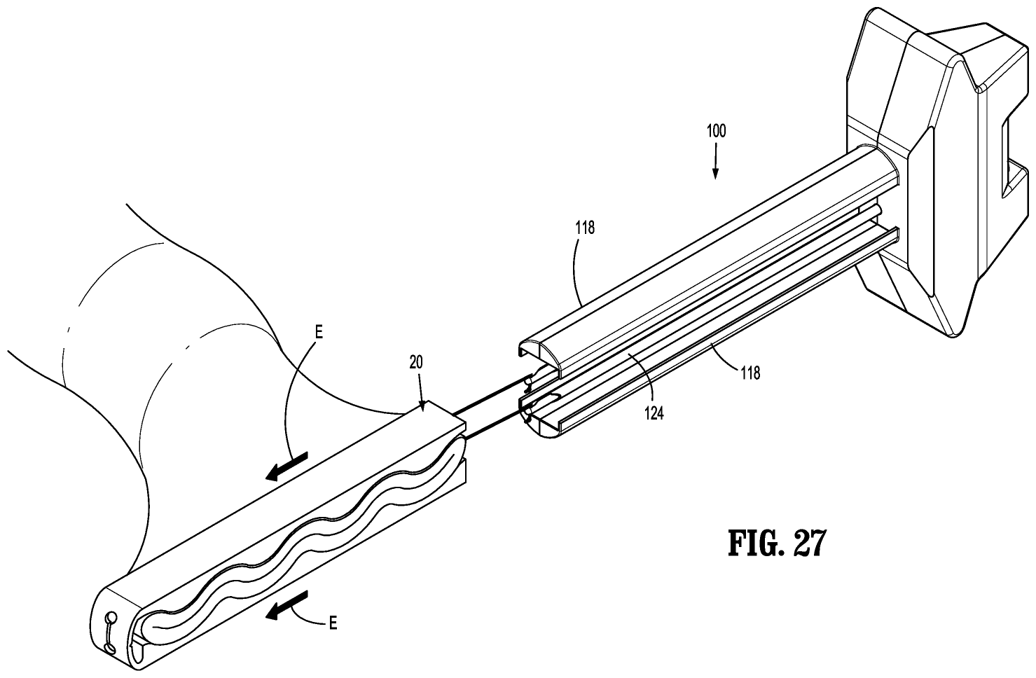

FIGS. 1-3 illustrate aspects of the clamp member 20 which includes a first jaw 22, a second jaw 24, and a hinge 26 (FIG. 26). The hinge 26 couples the first jaw 22 with the second jaw 24 such that the clamp member 20 can pivot between the open and clamped positions. In aspects of the disclosure, the hinge 26 is integrally formed with the first and second jaws 22 and 24 and forms a living hinge. Alternately, the hinge 26 can be in the form of any known coupling member that couples the first jaw 22 with the second jaw 24 to facilitate pivoting movement of the first jaw 22 in relation to the second jaw 24 between the open and clamped positions. For example, the hinge 26 can be in the form of a pivot pin (not shown). In aspects of the disclosure, the hinge 26 defines spaced through bores 28. In some aspects of the disclosure, the hinge includes a slot 29 that extends between the spaced through bores 28 and adds flexibility to the hinge 26.

Each of the first and second jaws 22 and 24 includes an outer surface 30 and an inner clamping surface 32. In aspects of the disclosure, the outer surfaces 30 of the first and second jaws 22 and 24 are substantially planar although other configurations are envisioned. The first and second jaws 22 and 24 may have a rectangular cross-section although other configurations are envisioned. The inner clamping surfaces 32 of the first and second jaws 22 and 24 each have a sinusoidal configuration that defines hills 34 and valleys 36 (FIG. 3). When the clamping member 20 is in the clamped position (FIG. 14), the clamping surfaces 32 of the first and second jaws 22 and 24 are in juxtaposed opposition with the hills 34 on the clamping surface 32 of the first jaw 22 received within the valleys 36 of the second jaw 24 and the hills 34 on the clamping surface 32 of the second jaw 24 received within the valleys 36 of the clamping surface 32 of the first jaw 24.

In aspects of the disclosure, each of the hills 34 on the clamping surfaces 32 of the first and second jaws 22 and 24 defines a through bore 38 that extends in a direction that is parallel to the longitudinal axis “X” (FIG. 14) of the clamp member 20. The through bores 38 in the first jaw 22 are aligned with each other to define a longitudinal passage that extends through first jaw 22. Similarly, the through bores 38 in the second jaw 24 are aligned with each other to define a longitudinal passage that extends through second jaw 24. The through bores 38 on the first and second jaws 22 and 24 are aligned with the through bores 28 in the hinge 26 to define two passages that extend longitudinally through the clamp member 20. In aspects of the disclosure, a slot 40 that communicates with each of the through bores 38 is defined in each of the hills 34 in each of the first and second jaws 22 and 24. The slots 40 allow portions of the first and second jaws 22 and 24 that define the through bores 38 to be deformed outwardly as described in further detail below.

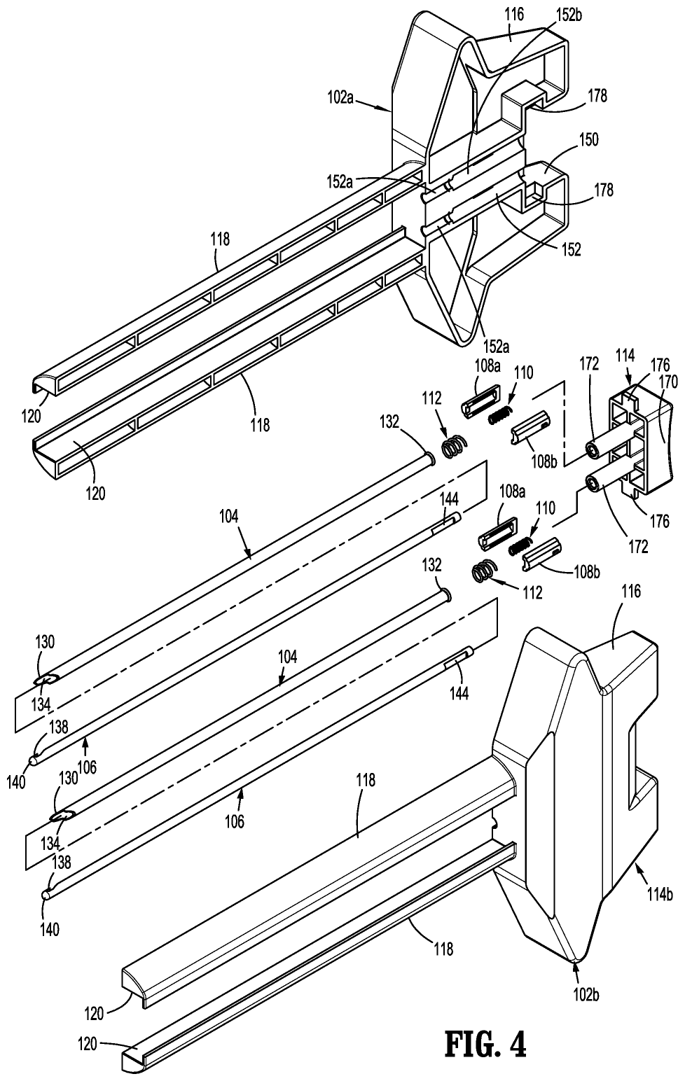

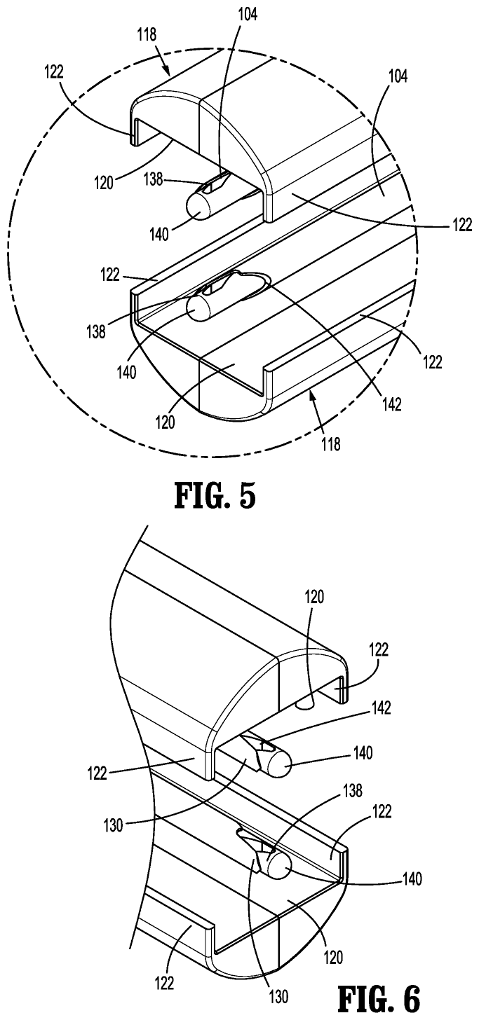

FIGS. 4-6 illustrate the housing assembly 100 which includes a body 102 (FIG. 1), hollow needles 104, suture passers 106, a coupling member 108, first biasing members 110, second biasing members 112, and an actuator 114. In aspects of the disclosure, the body 102 is formed from body half-sections 102a and 102b that are coupled together to form the body 102. The body 102 includes a grip portion 116 and spaced guide portions 118 that extend distally from the grip portion 116. Each of the guide portions 118 includes an inner guide surface 120 that faces the guide surface 120 of the other guide portion 118. The guide portions 118 also include longitudinally extending guide walls 122 that extend from the respective guide surface 120 of the guide portion 118 towards the other guide portion 118 of the body 102 to define a channel 124 between the guide portions 118. The channel 124 has a configuration that corresponds to the configuration of the clamp member 20 when the clamp member 20 is in the clamped position such that the clamp member 20 (FIG. 1) can be slid into the channel 124. In aspects of the disclosure, the guide surfaces 120 are angled towards each other in a proximal direction such that the distance between the guide surfaces 120 decreases slightly towards the proximal ends of the guide portions 118. In such a configuration, the clamping member 20 will be more tightly clamped as the clamp member 20 is inserted further into the guide channel 124.

Each of the needles 104 includes a distal portion and a proximal portion. The distal portion has a sharpened distal end 130 that is configured to pierce tissue. The proximal portion of each of the needles 104 includes a transversely extending flange 132. Each of the needles 104 is hollow and defines a longitudinal through bore 134 that receives one of the suture passers 106.

Each of the suture passers 106 has a distal portion and a proximal portion. The distal portion of each of the suture passers 106 includes a hook portion 138 and a blunt distal end 140 that is positioned distally of the hook portion 138. The hook portion 138 defines a recess 142 (FIG. 8) that is configured to receive a portion of a suture “S” (FIG. 26). The proximal portion of each of the suture passers 106 includes a reduced diameter portion 144 that is configured to be coupled to the coupling member 108 as described in further detail below.

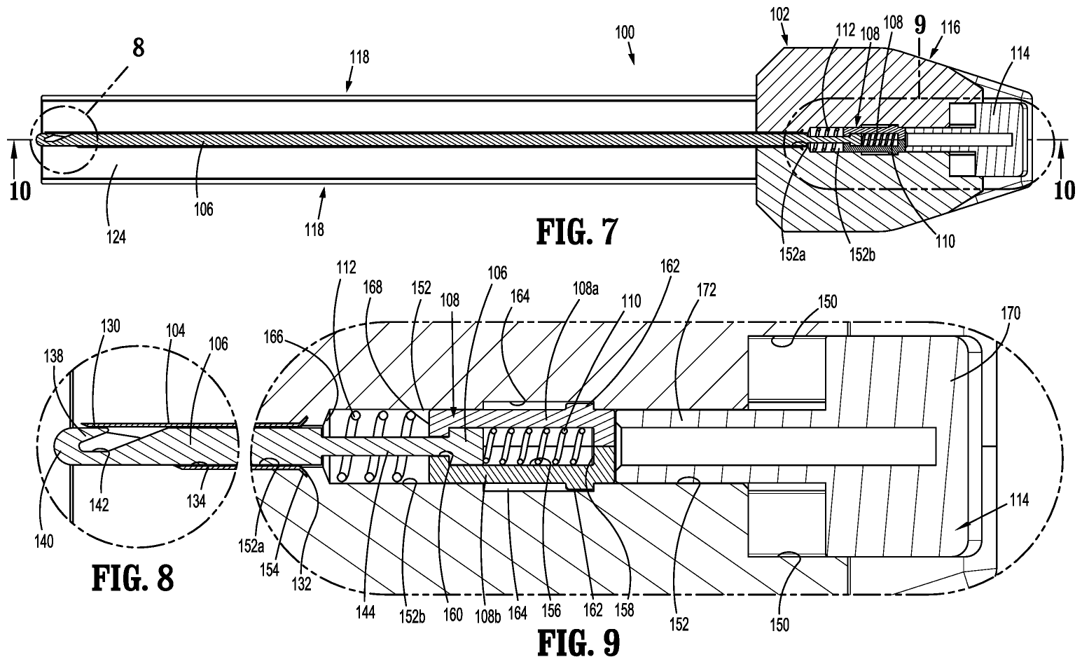

FIGS. 4, 7, and 9 illustrate a proximal portion of the housing assembly 100 including the grip portion 116. The grip portion 116 defines a proximal cutout 150 and spaced through bores 152 (FIG. 4). Each of the through bores 152 is stepped and has a smaller diameter distal portion 152a (FIG. 9) and a larger diameter proximal portion 152b (FIG. 9). The distal portion 152a of each of the spaced through bores 152 receives the proximal portion of the one of the needles 104. The flanges 132 of the needles 104 are received within slots 154 (FIG. 9) defined in the grip portion 116 to axially fix the needles 104 to the grip portion 116. When the needles 104 are secured to the grip portion 116 of the body 102 of the housing assembly 100, the needles 104 are positioned between the respective guide portions 118 of the body 102 of the housing assembly 100 within the guide channel 124 such that the longitudinal through bores 134 defined by the needles 104 are longitudinally aligned and communicate with the proximal portions 152b of the respective through bores 152 in the grip portion 116.

The suture passers 106 extend through the through bores 152 in the grip portion 116 of the body 102 of the housing assembly 100 and through the longitudinal through bores 134 of the needles 104 such that the hook portions 140 of the suture passers 106 are positioned adjacent the distal ends of the needles 104 (FIG. 8). The coupling members 108 are secured to the proximal portions of the suture passers 106. In aspects of the disclosure, each of the coupling members 108 is formed from half-sections 108a and 108b (FIG. 4) that are coupled together to define a cavity 156 (FIG. 9) within the coupling member 108. Each of the cavities 156 of the coupling members 108 is defined by a closed proximal wall 158 (FIG. 9) and a distal wall that defines an opening 160. The openings 160 of the coupling members 108 receive the reduced diameter portions 144 of the suture passers 106 but are sized to prevent passage of the proximal portion 106a of the suture passer 106 such that the coupling members 108 are slidably secured to the suture passers 106 and movable in relation to the suture passers 106 between a retracted position (FIG. 9) and an advanced position (FIG. 20).

The first biasing members 110 are positioned within the cavities 156 of the coupling members 108 between the proximal walls 158 of the coupling members 108 and the respective proximal ends of the suture passers 106. The first biasing members 110 urge the coupling members 108 in relation to the suture passers 106 towards their retracted positions (FIG. 9). In aspects of the disclosure, the first biasing members 110 include coil springs although other types of biasing members are envisioned.

Each of the coupling members 108 includes outer protrusions 162 (FIG. 9) that are received within channels 164 defined along the proximal portion 152b of the through bores 152. Receipt of the protrusions 162 within the channels 164 allows movement of the coupling members 108 within the proximal portion 152b of the through bores 152 between retracted and advanced positions.

The second biasing members 112 are received within the proximal portions 152b of the respective through bores 152 between a distal wall 166 defining a portion of the through bore 152 and a distal surface 168 of the respective coupling member 108. The second biasing members 112 urge the coupling members 108 towards their retracted positions (FIG. 9) to urge the suture passers 106 to their retracted positions within the needles 104. As illustrated in FIG. 8, when the suture passers 106 are in their retracted positions within the needles 104, the blunt distal ends 140 of the suture passers 106 are positioned distally of the sharpened distal ends 130 of the needles 104 and the recesses 142 defined by the hook portions 138 of the sutures passers 106 are covered by the needles 104. In aspects of the disclosure, the second biasing members 112 include coil springs although other types of biasing members are envisioned.

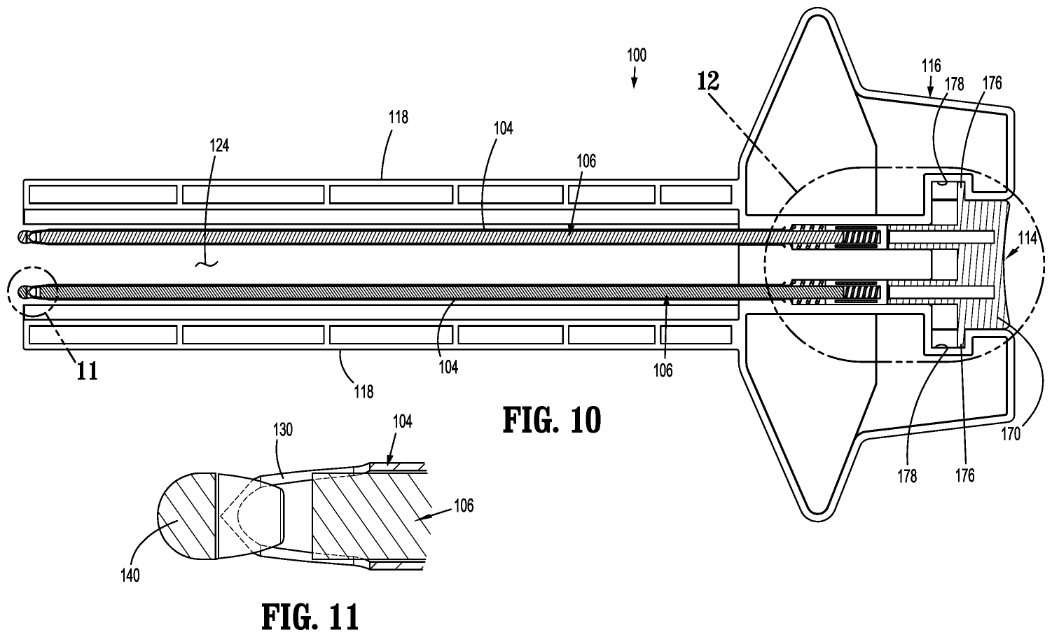

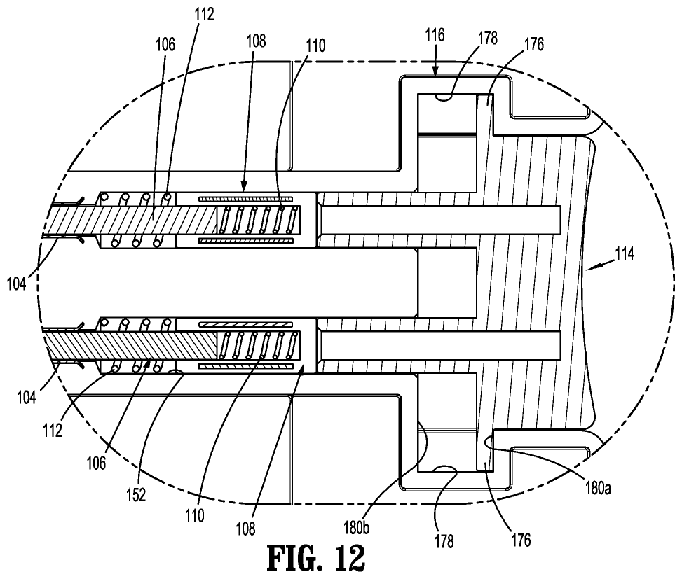

FIGS. 9-12 illustrate the actuator 114 which includes a finger engagement member 170 and spaced distal extensions 172. The finger engagement member 170 is received within the cutout 150 in the grip portion 116 of the body 102 of the housing assembly 100 such that each of the spaced distal extensions 172 is received within the proximal portion 152b of one of the through bores 152. The distal extensions 172 engage proximal surfaces of the coupling members 108. The finger engagement member 170 includes tabs 176 (FIG. 12) that are received within recesses 178 formed in the grip portion 116 of the body 102 of the housing assembly 100 to retain the actuator 114 on the grip portion 116. The actuator 114 is movable within the cutout 150 from a retracted position (FIG. 9) to an advanced position to advance the coupling members 108 within the through bores 152 of the grip portion 116. When the actuator 114 moves between its retracted and advanced positions, the tabs 176 on the actuator 114 move within the recesses 178. Engagement of the tabs 176 with proximal and distal walls 180a and 180b (FIG. 12) of the grip portion 116 defines the retracted and advanced positions of the actuator 114. As illustrated (FIG. 9), the second biasing members 112 urge the coupling members 108 towards their retracted positions to urge the actuator 114 towards its retracted position and move the suture passers 106 to an intermediate position as described in further detail below.

FIGS. 13-15 illustrate the clamp member 20 as tissue “T” is clamped between the first and second jaws 22 and 24 of the clamp member 20. The tissue “T” is a section of a hollow vessel “V” to be anastomosed. In order to clamp the tissue “T” within the clamp member 20, the clamp member 20 is opened and positioned around the vessel “V” and then moved in the direction of arrow “A” in FIG. 14 to the clamped position to compress the vessel “V” between the jaw members 22 and 24. When the vessel “V’ is clamped between the jaws 22 and 24 of the clamp member 20, the tissue “T” of the vessel “V” is compressed between the sinusoidal clamping surfaces 32 between the hills 34 and the valleys 36 of the clamp member 20 to assume a sinusoidal configuration.

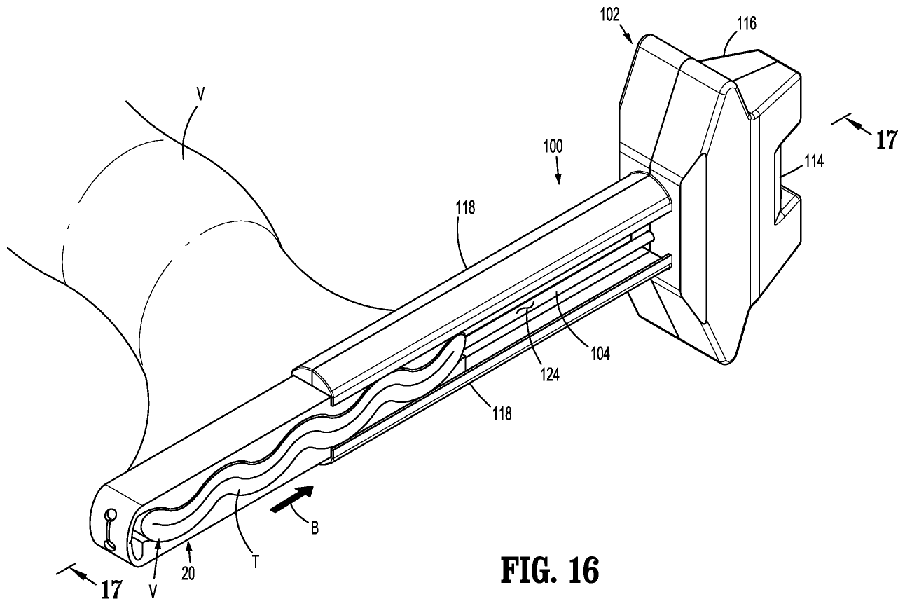

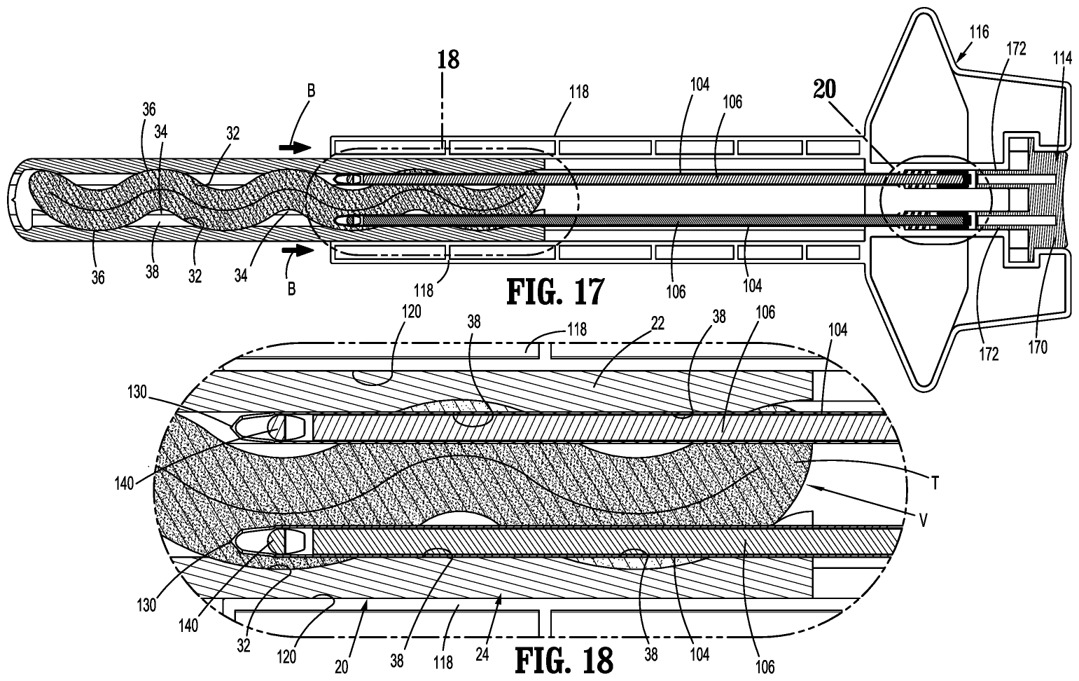

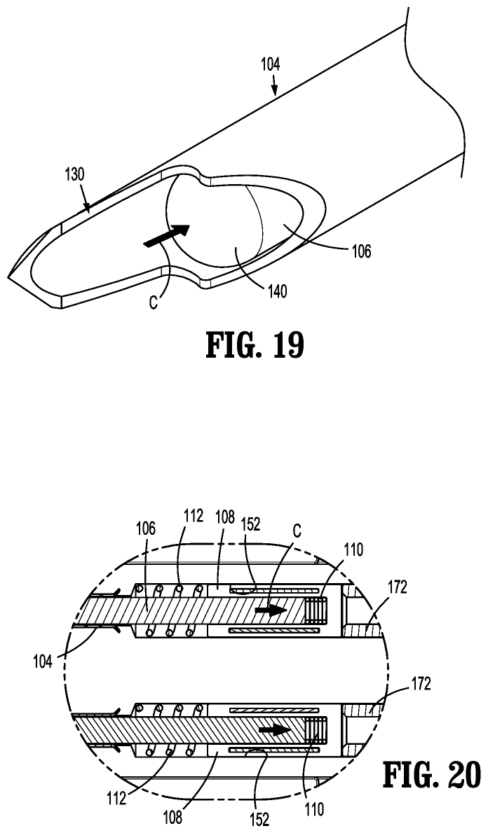

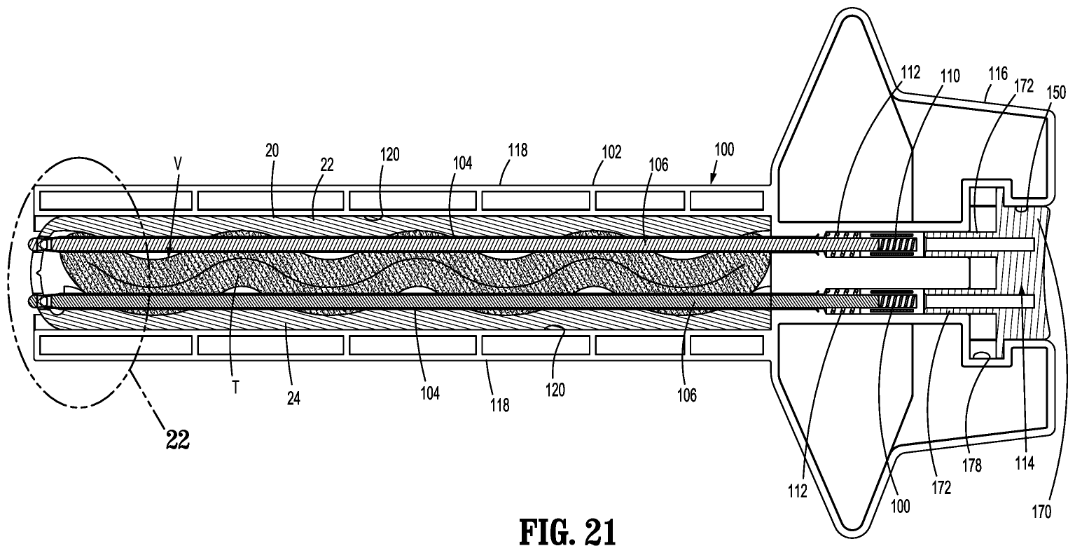

FIGS. 15-20 illustrate the clamp member 20 as the clamp member 20 is slid into the guide channel 124 defined between the guide portions 118 of the body 102 of the housing assembly 100. The channel 124 is dimensioned to retain the clamp member 20 in the clamped position. As the clamp member 20 is inserted into the guide channel 124 in the clamped position in the direction of arrows “B” in FIGS. 15-17, the clamp member 20 is guided along the inner guide surfaces 120 and outer guide walls 122 of the guide portions 118 until the clamp member 20 is fully received within the channel 124 (FIG. 21). As the clamp member 20 moves through the guide channel 124, the needles 104 and the suture passers 106 are received in and pass through the through bores 38 in the first and second jaws 22 and 24 between the hills 34 and the valleys 36 in the clamping surfaces 32. As the needles 104 and suture passers 106 pass through the through bores 38, the blunt distal ends of the suture passers 106 initially engage the tissue “T” of the vessel “V”. When the blunt distal ends 140 of the suture passers 106 engage the tissue “T”, a force is exerted on the blunt distal end 140 in the direction of arrows “C” in FIGS. 19 and 20. This force on the suture passers 106 urges the suture passers 106 proximally within the needles 104 to their retracted positions against the bias of the biasing members 110 to retract the suture passers 106 into the needles 104 and expose the sharpened distal end 130 of the needles 104 (FIG. 19). With the sharpened distal ends 130 of the needles 104 exposed, the sharpened distal ends 130 of the needles 104 penetrate and pass through the tissue “T” of the vessel “V” (FIG. 18).

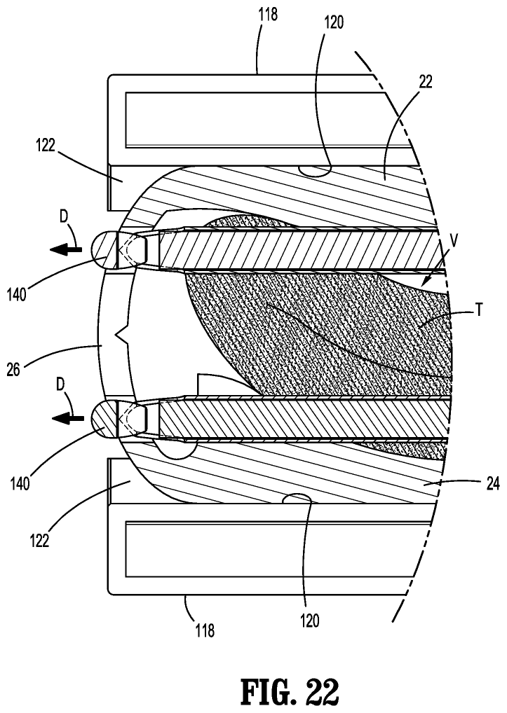

FIGS. 21 and 22 illustrate the needles 104 and suture passers 106 as the needles 104 and suture passers 106 pass through the through bores 28 of the hinge 26 of the clamp member 20. When the sharpened distal ends of the needles 104 eventually pass through the through bores 28 in the hinge 26 and extend from the clamp member 20 (FIG. 21), the force on the suture passers 106 is removed. As such, the biasing members 110 advance the suture passers 106 within the needles 104 in the direction indicated by arrows “D” in FIG. 22 back to their intermediate positions. In their intermediate positions, the blunt distal ends 140 of the suture passers are positioned distally of the sharpened distal ends 130 of the needles 104 to shield the needles 104.

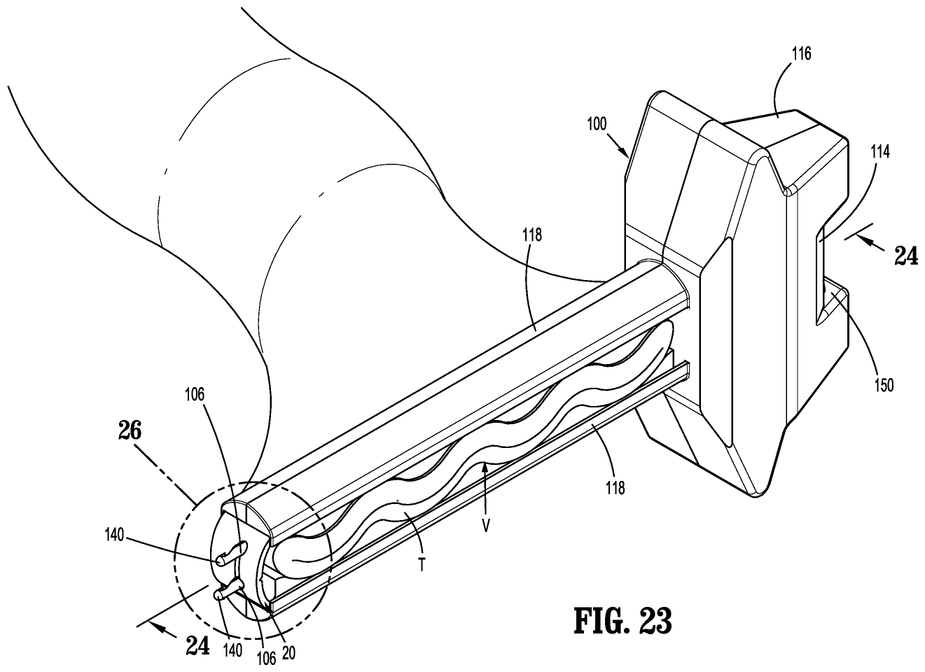

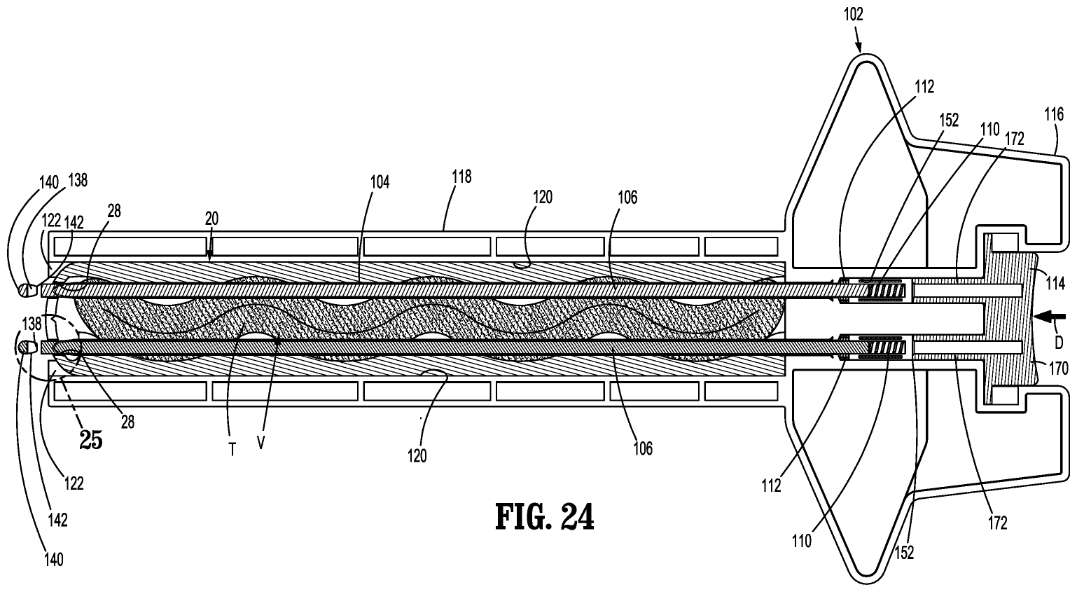

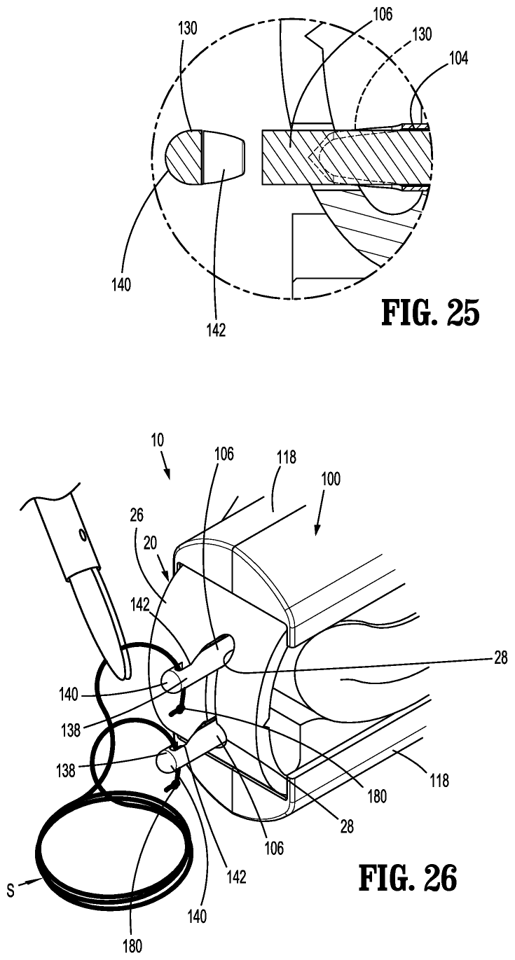

FIGS. 23-25 illustrate the housing assembly 100 with the clamp member 20 inserted into the body 102 of the housing assembly 100 and the suture passers 106 moved to their advanced positions. When the clamp member 20 is fully inserted into the guide channel 124 defined between the guide portions 118 of the body 102 of the housing assembly 100, the actuator 114 is depressed in the direction indicated by the arrow “D” in FIG. 24 to move the suture passers 106 within the needles 104 to their advanced positions (FIG. 26). As the actuator 114 is depressed, the distal extensions 172 of the actuator 114 (FIG. 24) engage and advance the coupling members 108 within the through bores 152 of the grip portion 116 against the urging of the biasing member 112 to advance the suture passers 106 to their advanced positions. In their advanced positions, the hook portions 130 of the suture passers 106 are advanced from the distal portion of the needles 104 to expose the recesses 142 defined by the hook portions 138.

FIG. 26 illustrates the distal portion of the passer device 10 as a suture “S” is positioned within the recesses 142 defined by the hook portions 138. When the suture passers 106 are in their advanced positions, ends of the suture “S” are positioned within the recesses 142 of the suture passers 106. In aspects of the disclosure, the suture “S” includes a knot 180 to facilitate engagement by the hook portions 138 of the suture passers 106. Once the ends of the suture “S” are received within the recesses 142 of the suture passers 106, the actuator 114 (FIG. 24) is released to allow the second biasing member 112 (FIG. 24) to return the suture passers 106 within the needles 104 to their intermediate positions. In their intermediate positions, the ends of the suture “S” are drawn into the needles 104. After the ends of the suture “S” are drawn into the needles 104, the clamp member 20 is withdrawn from the channel 124 defined by the guide portions 118 of the body 102 of the housing assembly 100 (FIG. 27) in the direction indicated by arrows “E” in FIG. 27. As the clamp member 20 is withdrawn from the channel 124, the needles 104 and the suture passers 106 are withdrawn through the through bores 28 and 38 (FIG. 24) of the jaws 22 and 24 of the clamp member 20 through the tissue “T” of the vessel “V” to place a purse string suture within the tissue “T” about the vessel “V”.

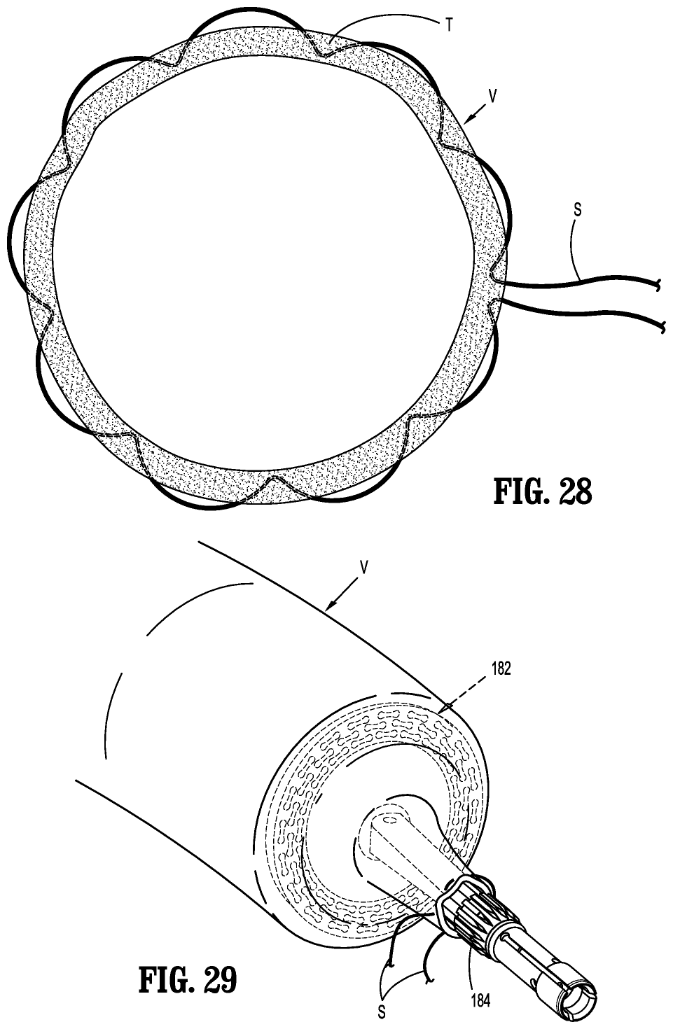

FIG. 28 illustrates the vessel “V” after the clamp member 20 is removed from the channel 124 of the body 102 of the suture housing assembly 100 and the clamp member 20 is removed from the vessel “V”. As illustrated, as the suture “S” is withdrawn through the tissue “T” of the vessel portion “V” with the suture passer 106, the suture “S” is threaded through the tissue “T” about the vessel “V” in purse-string fashion.

During an anastomosis procedure, after the purse-string suture “S” is placed in the tissue “T” about the vessel “V”, a surgical instrument, e.g., an anvil assembly 182 of a surgical stapling device (not shown), is inserted into the vessel “V” with an anvil shaft 184 of the anvil assembly 182 extending from the vessel “V”. In order to secure the vessel “V” about the anvil shaft 184, the ends of the suture “S” are pulled to cinch the suture “S” and the vessel “V” about the anvil shaft 184.

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. A purse string suture passer device comprising:
a clamp member including a first jaw, a second jaw, and a hinge coupling the first jaw to the second jaw, the first jaw and the second jaw movable in relation to each other about the hinge such that the clamp member is movable between open and clamped positions, each of the first and second jaws including a clamping surface having a sinusoidal configuration including hills and valleys, the hills of the first jaw being aligned with the valleys of the second jaw and the valleys of the first jaw being aligned with the hills of the second jaw when the clamp member is in the clamped position, each of the hills of the first and second jaws defining a longitudinal through bore that extends though the hills to define a longitudinal passage through each of the first and second jaws; and
a housing assembly including:
a body having a grip portion and first and second guide portions that extend distally from the grip portion, the first and second guide portions being spaced from each other to define a guide channel between the guide portions that is dimensioned to receive the clamp member when the clamp member is in the clamped position;
first and second hollow needles fixedly secured to and extending distally from the grip portion within the guide channel; and
first and second suture passers, the first suture passer positioned within the first hollow needle and the second suture passer being positioned within the second hollow needle, each of the first and second suture passers including a distal hook portion that defines a recess;
wherein the first hollow needle is aligned with the longitudinal passage in the first jaw and the second longitudinal needle is aligned with the longitudinal passage in the second jaw such that when the clamp member is received within the guide channel defined between the first and second guide portions, the first hollow needle extends through the first jaw and the second hollow needle extends through the second jaw.

2. The purse string suture passer device of claim 1, wherein the first and second suture passers are movable within the first and second needles, respectively, from an intermediate position in which the recesses defined by the distal hook portions are covered by the needle to an advanced position in which the recesses defined by the distal hook portions are uncovered and positioned distally of the needle.

3. The purse string suture passer device of claim 2, wherein the first and second suture passers are movable within the first and second needles, respectively, from the intermediate position to a retracted position in which the distal portions of the first and second needles are positioned distally of the first and second suture passers.

4. The purse string suture passer device of claim 3, wherein the distal portion of each of the first and second suture passers includes a sharpened distal end that is configured to pierce tissue.

5. The purse string suture passer device of claim 4, wherein the housing assembly includes first biasing members positioned within the grip portion to urge the first and second suture passers towards the intermediate position.

6. The purse string suture passer device of claim 5, wherein the housing assembly includes an actuator supported on the grip portion, the actuator being movable from a retracted position to an advanced position to move the first and second suture passers from their intermediate positions to their advanced positions.

7. The purse string suture passer device of claim 1, wherein each of the first and second guide portions includes an inner guide surface, the inner guide surfaces of the first and second guide portions defining the guide channel.

8. The purse string suture passer device of claim 7, wherein each of the guide portions includes guide walls positioned on opposite sides of the guide surface, the guide walls and the guide surfaces of the first and second guide portions defining the guide channel.

9. The purse string suture passer device of claim 8, wherein the guide surfaces of the first and second guide portions are angled towards each other in a proximal direction.

10. The purse string suture passer device of claim 5, further including a coupling member secured to the proximal portion of each of the first and second suture passers, each of the coupling members defining a cavity, wherein the first biasing members are positioned within cavities of the coupling members and the coupling members are movable in relation to the first and second suture passers between retracted and advanced positions.

11. A clamp member comprising:
a first jaw, a second jaw, and a hinge coupling the first jaw to the second jaw, the first jaw and the second jaw movable in relation to each other about the hinge such that the clamp member is movable between open and clamped positions, each of the first and second jaws including a clamping surface having a sinusoidal configuration including hills and valleys, the hills of the first jaw being aligned with the valleys of the second jaw and the valleys of the first jaw being aligned with the hills of the second jaw when the clamp member is in the clamped position, each of the first and second jaws defining a longitudinal through bore that extends though the hills to define a longitudinal passage through each of the first and second jaws.

12. The clamp member of claim 11, wherein the hinge is a living hinge that is integrally formed with the first and second jaws.

13. The clamp member of claim 12, wherein each of the first and second jaws has a rectangular cross-sectional configuration.

14. A suture passer housing assembly comprising:
a body having a grip portion and first and second guide portions that extend distally from the grip portion, the first and second guide portions spaced from each other to define a guide channel between the first and second guide portions that is dimensioned to receive a clamp member;
first and second hollow needles fixedly secured to and extending distally from the grip portion within the guide channel; and
first and second suture passers, the first suture passer positioned within the first hollow needle and the second suture passer being positioned within the second hollow needle, each of the first and second suture passers including a distal hook portion that defines a recess.

15. The suture passer housing assembly of claim 14, wherein the first and second suture passers are movable within the first and second needles, respectively, from an intermediate position in which the recesses defined by the distal hook portions are covered by the needle to an advanced position in which the recesses defined by the distal hook portions are uncovered and positioned distally of the needle.

16. The suture passer housing assembly of claim 15, wherein the first and second suture passers are movable within the first and second needles, respectively, from the intermediate position to a retracted position in which the distal portions of the first and second needles are positioned distally of the first and second suture passers.

17. The suture passer housing assembly of claim 16, wherein the distal portion of each of the first and second suture passers includes a sharpened distal end that is configured to pierce tissue.

18. The suture passer housing assembly of claim 17, further including first biasing members positioned within the grip portion, the first biasing members positioned to urge the first and second suture passers towards the intermediate position.

19. The suture passer housing assembly of claim 18, further including an actuator supported on the grip portion, the actuator being movable from a retracted position to an advanced position to move the first and second suture passers from their intermediate positions to their advanced positions.

20. The suture passer housing assembly of claim 14, wherein each of the first and second guide portions includes an inner guide surface, the inner guide surfaces of the first and second guide portions defining the guide channel.
